(19) United States
(12) Patent Application Publication
(10) Pub. No.: US 2003/0175345 A1
(43) Pub. Date: Sep. 18, 2003

(54) Isoflavone composition for oral delivery

(76) Inventors: Michael P. Hite, Seattle, WA (US); Stephen J. Turner, Covington, WA (US); Catherine Federici, Seattle, WA (US)

Correspondence Address: DAVIS WRIGHT TREMAINE, LLP, 2600 CENTURY SQUARE, 1501 FOURTH AVENUE, SEATTLE, WA, 98101-1688, US

(21) Appl. No.: 10/313,129

(22) Filed: Dec. 6, 2002

Related U.S. Application Data
(60) Provisional application No. 60/339,887, filed on Dec. 6, 2001.

(51) Int. Cl. A61K 9/22; A61K 31/7048; A61K 31/353

(52) U.S. Cl. 424/468; 514/27; 514/456

(57) ABSTRACT

A controlled release delivery system composition and method for oral administration of an isoflavone is disclosed

### CROSS REFERENCE TO RELATED APPLICATION

This application claims the benefit of U.S. Ser. No. 60/339,887, filed on Dec. 6, 2001, incorporated herein in its entirety.

### BACKGROUND

Active nutritional ingredients can elicit beneficial physiological responses when proper doses are taken. One such nutritional ingredient that has stimulated great interest in recent years is that of the isoflavones, active components found in legumes, the most notable being soybeans, and in other plants such as red clover. Isoflavones, which are heterocyclic phenols, are understood to include the soy compounds genistin, daidzin and glycitein, as well as biochanin A, equol, formononetin, and o-desmethylangolensin and natural derivatives thereof. These compounds and their aglycone or de-methylated aglycone forms, such as genistein and daidzein, are believed to have similar activities once they are ingested. They are sometimes referred to as phytoestrogens.

The benefits of isoflavones are broad and varied. Studies have shown that they decrease serum cholesterol, and therefore enhance heart health. Anderson J W, Johnstone B M, and Cook-Newell M E. 1995, Meta-analysis of the effects of soy protein intake on serum lipids, New England J. Med. August 3, 333(5): 276-282. It has also been shown that isoflavones are molecularly structurally similar to estrogen and, therefore, have demonstrated mild estrogenic activity. One of the benefits of this is that they alleviate menstrual cycle and menopausal symptoms. Washburn S, Burke G L, and Morgan T, et al. 1999, Effect of soy protein supplementation of serum lipoproteins, blood pressure, and menopausal symptoms in perimenopausal women, Menopause. Spring 6(1): 7-13 and Xu S, Duncan A M, Merz B E, et al. 1998, Effects of soy isoflavones on estrogen and phytoestrogen metabolism in premenopausal women, Cancer Epidemiology Biomarkers Prev. 7(12): 1101-8. Also, most probably a result of their estrogenic activity, isoflavones help maintain bone mass in post-menopausal women. Potter S M, Baum J A, and Teng H, et al., Soy protein and isoflavones: their effects on blood lipids and bone density in post-menopausal women, Nutrition 68(6 Suppl): 1375S-1379S. Isoflavones alone may also reduce or prevent various symptoms related to the onset and duration of menopause, including hot flashes and osteoporosis.

Isoflavones may also be cancer-preventing agents. This may be due to their ability to lower blood estrogen levels and alter estrogen metabolism. Two primary isoflavones are daidzein and genistein. Several studies have found genistein to have particularly potent anticancer activity. Isoflavones can be used alone to treat or prevent breast cancer, prostate cancer, skin cancer, and colon cancer or as mechanism inhibitors. Isoflavones alone may also be effective in certain cardiovascular applications, including heart disease, reducing cholesterol-lipid levels, modulating angiogenesis, and other vascular effects. Moreover, isoflavones along have been implicated in reducing headaches, dementia, inflammation, and alcohol abuse, as well as immunomodulation.

The many published health benefits of soy protein and isoflavones has resulted in an increased interest in including them in the diet. They are generally included in the form of soy-based foods or isoflavone enhanced nutritional supplements. Nutritional supplementation has the advantage that it allows consistent dosing of isoflavones in spite of a diet that may or may not include isoflavone-rich foods.

The principle isoflavones in soy are genistein, daidzein, and their metabolites. Genistein has a hydroxy group in the 5 position, giving it three hydroxy groups, while daidzein has just two hydroxy groups. Isoflavones are members of the large flavonoid family of plant compounds which are in turn members of the larger group of plant constituents known as polyphenols. Isoflavones are not as ubiquitous in nature as other flavonoids such as flavones and flavonols, being found primarily in one subfamily of Leguminosae, the Papilionoideae family.

Past formulations cannot orally deliver isoflavones over an extended time period without requiring coating or granulation processes to achieve controlled release. Further, past formulations fail to provide mechanisms for pH control thereby rendering pH sensitive isoflavones, glycosides and soy proteins due to variations in gastrointestinal tract (GI) pH. Further, past formulations lack mechanisms of isolating the soy proteins, glycosides and isoflavones from enzymatic degradation or hydrolysis. Formulations utilizing granulation and coating technologies do not allow for complete release of the isoflavones from the dosage form. These and other limitations and problems of the past are solved by the present invention.

### SUMMARY OF THE INVENTION

The present invention provides controlled release delivery systems for oral administration of compositions extracted from vegetable matter and more particularly to phytochemicals, such as an isoflavone composition.

One embodiment of a controlled delivery system includes a hydrogel or modified matrix formed from an excipient of one or more hydrophilic polymers, for example, polysaccharides, galactomannan gums, resins, polyethylene derivatives or hydrolyzed proteins, either alone or in combination, in which is disposed isoflavone composition. Optionally, the delivery system includes one or more additional release modifying excipients from the same group of hydrophilic agents for the purpose of attenuating the release of the isoflavone composition with pH-specific or enzyme-specific agents, and optionally, one or more physiologically acceptable electrolytic substances included for the purpose of pH control.

In another embodiment, a process for making an extended release dosage form, such as a tablet or capsule, from a pre-blend including mixing an isoflavone composition with one or more polymers, gums, resins, polyethylene derivatives, or hydrolyzed proteins for the purpose of controlled release. Optionally, the delivery system includes one or more physiologically acceptable electrolytic substances for the purpose of regulating pH within the dosage form, and optionally, one or more nutritional additives included for concurrent administration.

Compositions for isoflavone nutritional and/or pharmaceutical products are described. Particularly, these compositions impart controlled release of the isoflavone active ingredients in an oral delivery form, methods for the production of these compositions, and methods of treatment using these compositions.

One embodiment of a controlled delivery system includes a matrix composed of one or more hydrophilic polymers, polysaccharides, galactomannan gums, polyethylene derivatives or hydrolyzed proteins, alone or in combination, in which is disposed isoflavones and their related intermediaries and metabolites. Optionally, the delivery system includes one or more additional hydrophilic polymers from the same group and, optionally, one or more physiologically acceptable salts included for the purpose of pH control.

The system generally includes a hydrophilic agent, an electrolyte, and an isoflavone composition, and may optionally include fillers, release modifying agents, desiccants, and flow agents.

In one embodiment, a delivery system for disclosed including a hydrophilic agent and the isoflavone composition.

In another embodiment, a delivery system is disclosed including a hydrophilic agent, an electrolytic agent, and the isoflavone composition.

In yet another embodiment, a delivery system is disclosed including a hydrophilic agent, a release modifying agent, and the isoflavone composition.

In yet a further embodiment, a delivery system is disclosed including a hydrophilic agent, release-modifying agent, electrolyte, and the isoflavone composition.

Isoflavone controlled release formulations have many advantages over the current art. Targeted and prolonged delivery of isoflavones allows for the optimal therapeutic benefit to the end user. By extending the time that the isoflavones are present within the gastrointestinal (GI) tract, their availability is prolonged, offering additional benefits and reduction in side effects such as those experienced by menopausal women who suffer from flushing and other symptoms. Additionally, the controlled delivery of isoflavones have cancer protective and therapeutic effects. Through the maintenance of a constant pH within the dosage form surrounding the isoflavone, an optimal microenvironment may be created, thereby maximizing stability of the isoflavone composition released into the GI tract.

Another advantage is that the system disclosed requires only dry blend and direct compression steps, thereby making it easily transferable to sites of manufacture and relying on only conventional tableting or encapsulation equipment for production. A further advantage of the system is that it is relatively independent of the isoflavone employed in formulation; therefore, targeted delivery of different forms of isoflavones is also possible.

One advantage of the present system is the controlled release of the isoflavone from the dosage form into the surrounding environment.

Another advantage of the present system is the maintenance of a constant pH within the dosage form itself.

Yet another advantage of the present system is the controlled exposure of the isoflavone within the dosage form to aqueous media.

Yet another advantage of the present invention is the minimal burst effect and near complete release of the isoflavone from the dosage form itself.

Yet another advantage of the present system is its amenability to the addition of other nutritional and/or pharmaceutical compounds within the dosage form for concurrent administration.

Yet another advantage of the system is that it is capable of extended release over a 24-hour period, thereby allowing for the once per day administration of isoflavone compositions.

The invention will best be understood by reference to the following detailed description of the preferred embodiment. The discussion below is descriptive, illustrative and exemplary and is not to be taken as limiting the scope defined by any appended claims.

### BRIEF DESCRIPTION OF SEVERAL VIEWS OF THE DRAWINGS

FIG. 1 shows the effects of hydrophilic agents on the controlled release of an isoflavone composition from monolithic tablets and a commercially available immediate release tablet.

FIG. 2 shows the effects of hydrophilic agents on the controlled release of an isoflavone composition from monolithic tablets and a commercially available extended release capsule.

FIG. 3 shows the effects of the addition of pH- and enzyme-sensitive agents on the controlled release of an isoflavone composition from monolithic tablets.

FIG. 4 shows the effects of the addition of electrolytic agents on the controlled release of an isoflavone composition from monolithic tablets.

FIG. 5 shows the effects of the addition of pH- and enzyme-sensitive agents and electrolytic agents on the controlled release of an isoflavone composition from monolithic tablets.

FIG. 6 shows the effects of the addition of pH- and enzyme-sensitive agents and electrolytic agents on the controlled release of an isoflavone composition from capsules.

FIG. 7 shows the effects of the addition of a vitamin blend on the controlled release of an isoflavone composition from capsules.

FIG. 8 shows the effects of the addition of a vitamin blend on the controlled release of an isoflavone composition from monolithic tablets.

### DETAILED DESCRIPTION OF THE PREFERRED EMBODIMENT

A delivery system is disclosed for the controlled release of an isoflavone composition into the surrounding environment. Controlled release delivery systems include those systems capable of site specific delivery, extended release, sustained release, delayed release, repeat action, prolonged release, bimodal release, pulsitile release, modified delivery, pH sensitive delivery, and/or target specific delivery, among others.

The delivery system includes a delivered component. The delivered component may include a phytochemical, such as a soy protein. Isoflavones are components of soy. Isoflavone compositions include soy isoflavones, their intermediates and metabolites, and their associated glycosides or protein constituents. “Isoflavone” includes genistein and daidzein, genistin and daidzin, malonyl, acetyl, glucoside, glycitin and aglycone forms of the isoflavones.

The delivery system may take a solid dosage form. A solid dosage form may take the form of a tablet, capsule, wafer, or sachet, and is not limited to an orally administered dosage form such as a tablet or capsule. The dosage form can be tableted or encapsulated, where the capsules may be gelatin, cellulose or vegetable capsules for oral delivery. The delivery system can be a readily manufacturable solid dosage form. In one aspect, the dosage form is in the form of a monolithic tablet or capsule. When a tablet or capsule, it may be administered orally, anally, and vaginally, among other routes.

The delivery system includes a delivery vehicle. A delivery vehicle, for example a homogenously distributed matrix, is made up of hydrophilic agents. Hydrophilic agents include swelling, viscosity increasing and gel strength enhancing agents. More particularly, the hydrophilic agent is selected from at least one of the group, but not limited to: a) a starch selected from the group consisting of corn, rice, or potato starch; b) a hydrophilic gum, polysaccharide or galactomannan selected from the group consisting of pectin, agar, dextran, carageenan, tragacanth gum, locust beam gum, acacia gum, guar gum, xanthan gum, ghatti gum, alginic acid or sodium alginate; c) a cellulose derivative selected from the group consisting of methylcellulose, carboxymethylcellulose, sodium starch glycollate, sodium or calcium carboxymethylcellulose, hydroxyethyl methylcellulose, hydroxypropyl methylcellulose, ethylcellulose, ethylhydroxy ethylcellulose, ethylmethylcellulose, hydroxyethylcellulose, cellulose acetate phthalate or microcrystalline cellulose; d) silica, aluminum silicate, magnesium silicate, aluminum magnesium silicate, sodium silicate or feldspar, e) aluminum hydroxide; f) a protein selected from the group consisting of gelatin or casein; and g) a polymer selected from the group consisting of acrylate, carboxypolymethylene, a polyalkylene glycol or polyvinylpyrrolidone. In one aspect, the hydrophilic polymers are selected from the group of cellulose derivatives such as microcrystalline cellulose (MCC), hydroxypropyl methylcellulose (HPMC), or hydroxypropyl cellulose (HPC), or from gums and polysaccharides such as guar gum or maltodextrin.

Optionally, the delivery system may include agents added to aid in gastric bypass or modify the release profile of the isoflavone composition due to pH-specific swelling characteristics or site-specific enzyme degradation within the GI tract. These agents may include but are not limited to at least one of alginate, polysaccharides such as such as gelatin or collagen, guar gum, xanthan gum, pectin, heterogeneous protein mixtures, and polypeptides. The polysaccharides may be pectin and/or an alginate salt, among others. The galactomannan gums may be guar gum, xanthan gum and/or locust bean gum, among others. The polyethylene derivatives may be polyethylene oxide (PEO) and/or polyethylene glycol (PEG), among others. The hydrolyzed proteins may be gelatin and/or collagen, among others.

The delivery system may include an electrolyte. The electrolyte may be at least one of sodium, potassium, or calcium salts, among others. Through the inclusion of physiologically acceptable electrolytes, the buffered environment allows reconstitution and release to occur under optimal pH conditions that eliminate or reduce hydrolysis of the associate glycosides or protein constituents. The interaction between electrolytes and a hydrophilic agent may allow not only the pH-independent release of the isoflavone composition, but also allows for the internal pH of the dosage form to remain constant. It is this constant internal pH that contributes to the stability of the isoflavone composition in-vivo.

The delivery system may include a desiccant. The desiccant may include, but is not limited to, sodium carboxymethylcellulose, calcium carboxymethylcellulose, colloidal silica dioxide, and combinations thereof. The disintegration agent may include, but is not limited to, croscarmellose sodium sold as Solutab™ available from Blanver Farmoquimica LTDA and crosprovidone (insoluble polyvinylpyrrolidone) sold as Kollidon CL™ available from BASF.

The delivery system may include flow and lubrication agents. The flow agents may include, but are not limited to, magnesium stearate and stearic acid.

In a first embodiment of the delivery system, the delivery system includes a swelling hydrophilic agent and an isoflavone composition. In one aspect, the various components of the first embodiment are homogonously distributed within a solid matrix dosage form. In another aspect, the various components of the first embodiment may not be homogonously distributed in the solid matrix dosage form, but rather be in a bi-layer tablet or multi tablet form. For example, a bi-layer tablet is a single tablet which consists of two parts of a tablet held together. Each part may contain the same or different delivered components. The delivery system allows for a controlled exposure of the isoflavone composition within the dosage form to an aqueous media by controlling the hydration rate of the dosage form via polymer disentanglement and matrix erosion. In one aspect, the delivery system may also include a physiologically acceptable electrolyte, a release modifying excipient such as a gum or polysaccharide, a desiccant, and flow or tubing agents, alone or in combination. Release modifying excipients, such as gums and polysaccharides, may be used to induce site-specific release through pH-specific swelling or site-specific enzymatic degradation. Flow or lubrication agents may be used to improve the manufacturability.

In one aspect of the embodiment, the isoflavone composition may be a pre-blend, which can be blended with other nutritional additives for concurrent administration. The pre-blend may include a carrier. The carrier may be, but is not limited to, soy proteins, monosaccharides or polysaccharides, such as maltodextrin, swellable polymers, such as hydroxypropyl methylcellulose, inert fillers, such as microcrystalline cellulose or di-calcium phosphate, or nutritional additives such as Vitamin E. In the aspect wherein a carrier is included, the carrier may function to assist in the controlled release of the isoflavone composition, to aid in the manufacturability of the dosage form, or to increase the stability of the dosage form.

In one aspect, the dosage form is a monolithic tablet created from a direct-compressible dry blend which does not require processes, such as enteric coating, granulation, or spray drying.

Release of the isoflavone composition into the surrounding environment may be accomplished through a rate-controlled hydration and subsequent swelling of hydrophilic agents. The release of the isoflavone composition is determined by the erosion rate and polymeric disentanglement of the swollen hydrophilic matrix. Without subscribing to a particular theory of kinetics, the swelling of the hydrophilic matrix is retarded by a plurality of layers of viscous gelled hydrophilic agents; these gel-states result from the interaction of the hydrophilic agents with the penetrating gastrointestinal fluid. While primarily erosion dependent, the gradual hydration and gelling reaction within the hydrophilic matrix allows for a highly reproducible, programmable release pattern. The programmability of the system allows for nearly any physiologically relevant release pattern to be accomplished. Mathematical treatment of the hydrophilic matrix swelling, erosion, and ensuing release of isoflavone composition can be determined, though each model will be representative of the particular components specific to each formulation. Some examples of the mathematical treatment of the hydrophilic matrix swelling, erosion and ensuing rate of release can be found in U.S. Pat. Nos. 5,783,212, 6,090,411, and 6,337,091 hereby incorporated by reference in their entirety. This can be accomplished without the need for undue experimentation.

The tablets described in U.S. Pat. Nos. 6,090,411 and 6,337,091 depend upon the presence of a readily ionizable active pharmaceutical compound to facilitate their mechanisms of control. The tablet of U.S. Pat. No. 6,337,091 employs differential swelling and diffusion suppression: the process of granulation allows for differential hydration of the matrix and the granulated active compound, thus controlling the granulated active's release from the dosage form. The diffusion of the active from the dosage form is further retarded by the on-going association-dissociation process involved in the reversible complexation of ionizable active compound with the polyionic matrix. The tablet of U.S. Pat. No. 6,090,411 employs salting-out phenomena to alter the competition for water between ionizable active compounds and the surrounding polymers and electrolytes, thus further retarding the release of the active compound in conjunction with a peripheral hardening reaction resulting from this competition for water of hydration. Isoflavone compositions are non-readily ionizable or non-ionizable. The lack of an exclusive ionizable compound within the isoflavone composition, the presence of multiple isoflavones and associated soy proteins prevent exclusive ionic interactions between the electrolytes, polymers, and polyionic matrices. Therefore, delivery systems for isoflavones rely upon mechanisms of diffusion and erosion to retard the release of the isoflavone composition.

Formulation specific to the physical characteristics of each isoflavone composition and the desired release profile can be accomplished through both theoretical and empirical means, allowing dissolution of the system and isoflavone composition release to occur in a specific physiologic region. Release of contents in a given region of the GI tract is accomplished by the hydrophilic matrix containing the isoflavone compositions segregating the isoflavones from the external environment until the desired physiologic region of release, which may be employed to achieve gastric bypass. Consideration of both the area and duration of release is essential in formulation so as to program the system with an appropriate ratio of components to ensure the desired release profile.

The homologous distribution of the isoflavone composition within the hydrophilic matrix provides protection from the fluctuations in pH and exposure to enzymatic degradation present in external environment. This isolation from the outside environment allows the isoflavone composition to be protected from hydrolytic and enzymatic reactions significantly longer than with conventional immediate release dosage forms.

In another embodiment, the delivery system includes a swelling hydrophilic agent, an isoflavone composition, and an electrolyte. When physiologically acceptable electrolytes are included into the delivery system, the electrolyte maintains an intra-dosage form pH irrespective of the external pH. This internal pH may be modified through the selection of electrolytes that are both physiologically-acceptable for human consumption and chemically appropriate to individual isoflavone compositions. An example of a non-acceptable electrolyte is CaCl₂. Such an environment may limit the exposure of the isoflavone composition to fluctuations in gastrointestinal pH, resulting in an increase in isoflavone stability prior to their release into the environment. This isolation from the outside environment prevents hydrolysis in the presence of acid and significantly enhances the stability and solubility over other dosage forms.

In another embodiment of the delivery system, the delivery system includes a swelling hydrophilic agent, an isoflavone composition, and a release modifying excipient. The addition of release modifying excipients, such as hydrophilic polymers or gums demonstrating pH or enzyme sensitivity, may be employed to alter the swelling or erosion characteristics of the matrix, such as the initiation of swelling or the rate of erosion of the matrix. These release modifying excipients function in combination with the hydrophilic agent to control the release of the isoflavone composition. These excipients may be employed to reduce the amount of exposure to the gastric environment by reducing matrix swelling during exposure to gastric pH or during the time the dosage form is expected to transit through the stomach and pylorus. These release modifying excipients may be selected for their in vivo degradation characteristics that occur in localized regions of the gastrointestinal tract. The release modifying agent, when used alone, may function as the hydrophilic agent. One example of this, among many, is that pectin mainly breaks down at the higher pH and enzyme rich environment of the large intestine, thus it can be employed alone as the hydrophilic agent if a greater proportion of lower intestinal tract delivery was desired. Another example among others it that gelatin largely breaks down in the small intestine. With regards to pharmaceutical controlled release formulations, the location of polymer breakdown is of special significance as bioavailability is determined by the amount of drug released within a given timeframe relative to a physiological site of absorption specific to that type of compound. When delivering an isoflavone composition, the inclusion of release modifying excipients whose swelling characteristics are pH dependent, specifically compounds that preferentially swell in environments above pH 1.0-1.5, is useful for the delivery of isoflavone compositions that are susceptible to structural changes when exposed to low pH. The low-pH environment will inhibit swelling, thus retarding both isoflavone release and acid-penetration into the dosage form. The inclusion of release modifying excipients whose erosion is enzyme-dependent, specifically compounds that degrade preferentially in the presence of lower intestinal tract enzymes, is also useful for the delivery of isoflavone compositions whose structure may be susceptible hydrolysis in environments proximal to the locations of such enzymes.

In a particular embodiment, the isoflavone is Novasoy™ available from the Archer Daniels Midland Company of Decatur, Ill.

In one embodiment, the dosage form disclosed is formed from a pre-blend. When a monolithic tablet, the pre-blend is mixed using dry-blend techniques known to those skilled in the art, and the dosage form is created using a direct compression process. Employing a pre-blend that is formed using dry-blend techniques is a significant improvement over the use of blends resulting from granulation, spheronization-extrusion, or other processes that might expose the isoflavone composition to solvents and high temperature and potentially lower the efficacy or detrimentally effect the powder characteristics of the isoflavone composition. Employing a pre-blend that is capable of forming a monolithic dosage form using only the techniques of direct-compression, in the case of a tablet, or high speed encapsulation, in the case of a capsule, is a significant improvement over manufacturing processes that require multi-stage compression, multiple geometrically-altered components or coatings.

Unless otherwise noted, all of the following embodiments are formulated through standard dry blend and directly compression with an appropriate lubricant such as magnesium stearate or stearic acid. In a particular embodiment, a formulation combining the isoflavone composition with a suitable hydrophilic agent such as HPMC, MCC, or PEO, in a ratio of 1.0:0.1 to 1:25 isoflavone to hydrophilic agent.

In yet another embodiment of the delivery system is a formulation comprising the isoflavone composition, hydrophilic agent, and a physiologically acceptable salt such as NaHCO₃, Na₂CO₃, or CaCO₃, in a ratio of 1.0:0.1:0.1 to 1:25:25 isoflavone composition to hydrophilic agent to salt.

In yet a further embodiment of the delivery system is a formulation comprising the isoflavone composition, a hydrophilic agent, and a release modifying agent such as pectin, sodium alginate alginic acid, or a gum such as xanthan gum, guar gum, locust bean gum, or tragacanth gum, in a ratio of 1.0:0.1:0.1 to 1:25:25 isoflavone composition to hydrophilic agent to release modifying agent.

In yet another embodiment of the delivery system is a formulation comprising the isoflavone composition, a hydrophilic agent, a release modifying agent, and a physiologically acceptable salt in a ratio of 1.0:0.1:0.1:0.1 to 1:25:25:25 isoflavone composition to hydrophilic agent to release modifying agent to electrolyte.

Another embodiment of the delivery system is a formulation comprising the isoflavone composition, a hydrophilic agent, a release modifying agent, a physiologically acceptable salt and a carrier in a ratio of 1.0:0.1:0.1:0.1:0.1 to 1:25:25:25:25 isoflavone composition to hydrophilic agent to release modifying agent to electrolyte to carrier.

In the following Examples 1-9, the isoflavone composition employed was Novasoy™, although other isoflavones compositions could equally well have been used.

### METHODS

In the Examples below, the formulations were prepared in accordance with the following methods. In these formulations, tablets were prepared using a method of dry blending and direct compression using a Carver hydraulic press or a rotary tablet press. Evaluations were performed using a USP Type II (paddle) dissolution apparatus.

Examples 1-8 were conducted by exposing the dosages to 900 mL 0.1N HCl for 2 hours at 50 RPM. After 2 hours, approximately 8.8 mL 10M NaOH was added to each vessel to buffer the acidic media, simulating the transition from the stomach through the pylorus into the intestinal tract. A reference standard was left in non-buffered media to provide a maximum absorption reading for the first two hour timepoints, from which the percentage of isoflavone composition released was derived. At the end of the dissolution period, the dosage form was crushed and homogenized within the buffered media for the purpose of enumerating the isoflavone composition remaining in the tablet. A sample was taken from the dissolution media after homogenization.

### Example 1

A monolithic tablet of approximately 520 mg or about 681 mg having a hydrophilic agent and an isoflavone concentrate was prepared as shown in Table 1. A commercial immediate release tablet of isoflavone concentrate (A1) was used as a control group. In this example, the isoflavone concentrate used is Novasoy™ and the hydrophilic agent employed is Hydroxypropyl methylcellulose (HPMC) or polyethylene oxide (PEO). Without being bound to a theory, the addition of the hydrophilic agent retards the release of the isoflavone concentrate from the dosage form. Stearic acid is included as a flow agent. Silica is included as a flow agent and a desiccant. Di-calcium phosphate (DCP) is included as a filler and flow agent. The filler present in the commercial tablet is unknown.

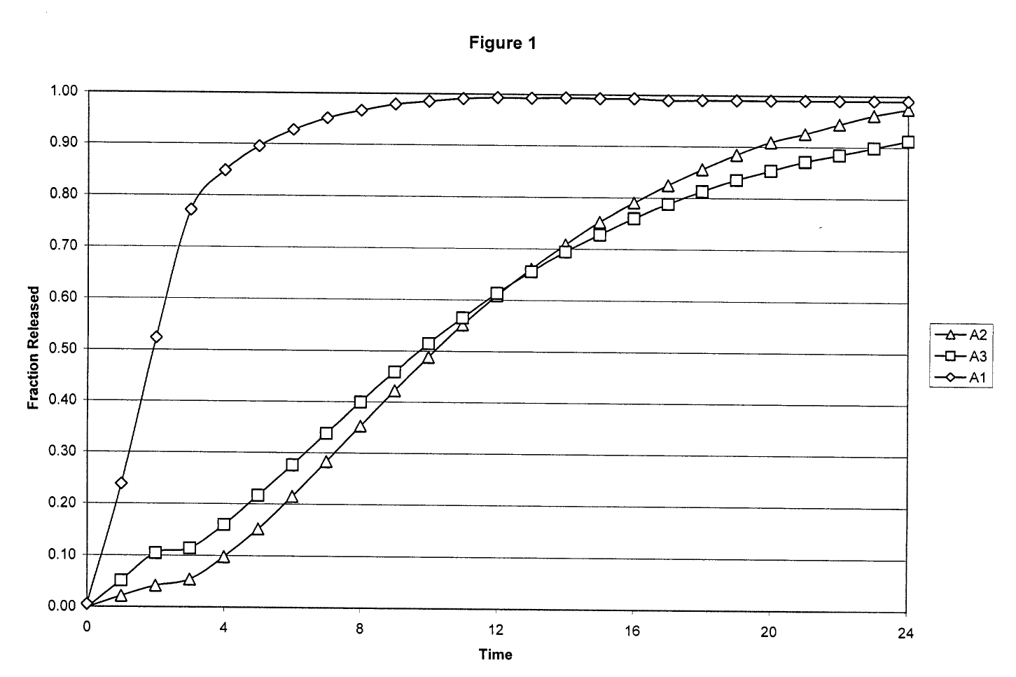

As shown in FIG. 1, the results of this example reflect a level of controlled release gained through the use of a matrix comprised of a hydrophilic agent and an isoflavone concentrate. This controlled release is shown over an extended duration. The hydrophilic agent is not limited to a particular type of hydrophilic agent, so long as sufficient matrix viscosity is achieved. This controlled release is shown through a more prolonged period of release of the majority of the contents than the control A1. The control A1 released 77% of its contents within the first 3 hours, whereas formulations A2 and A3 released 5% and 11% of their contents by hour 3, respectively.

TABLE 3

| Dosage | | | |
|---|---|---|---|
| Formulas (mg) | A1 (control) | A2 | A3 |
| Isoflavone concentrate | 125 | 250 | 250 |
| HPMC | n/a | 125 | 0 |
| PEO | n/a | 0 | 250 |
| DCP | n/a | 280 | 0 |
| Stearic Acid | n/a | 13 | 10 |
| Silica | n/a | 13 | 10 |
| Filler | 535 | 0 | 0 |
| Total Weight | 660 | 681 | 520 |

### Example 2

A monolithic tablet of approximately 943 mg having a hydrophilic agent, a release modifying agent, an electrolyte, and an isoflavone concentrate was prepared as shown in Table 2. A commercial extended release capsule of isoflavone granules was used as a control group B1. In this example, the isoflavone concentrate used is NovaSoy™ and the hydrophilic agent employed is Hydroxypropyl methylcellulose (HPMC), the release modifying agent is pectin, and the electrolyte is NaHCO₃. Without being bound to a theory, the addition of the hydrophilic agent will retard the release of the isoflavone concentrate from the dosage form. Stearic acid is included as a flow agent. Silica is included as a flow agent and a desiccant. Di-calcium phosphate (DCP) is included as a filler and flow agent.

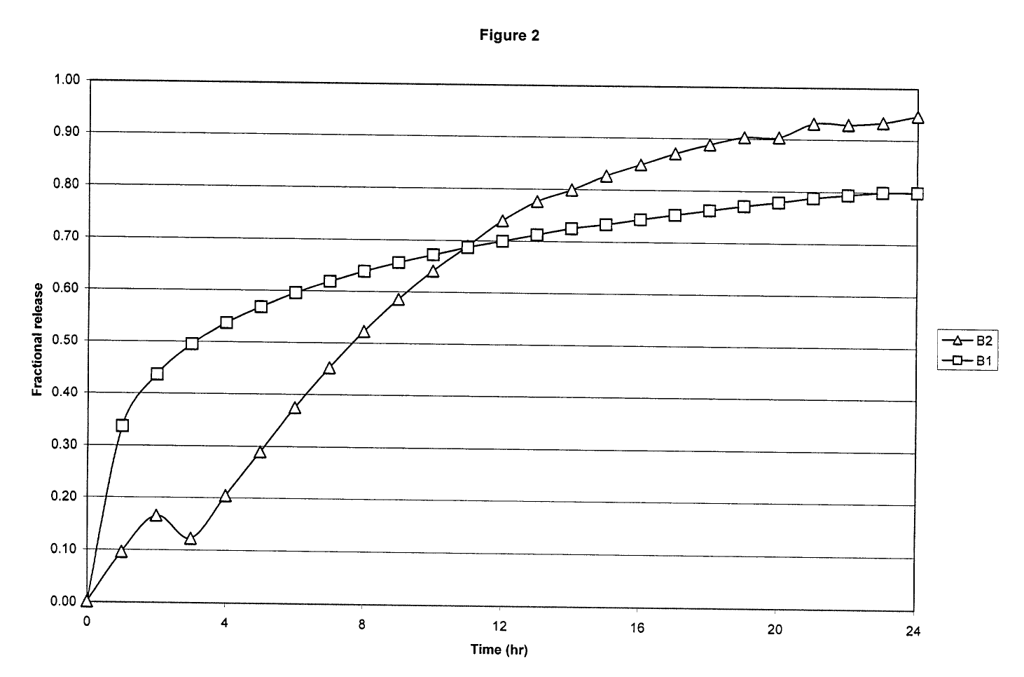

As shown in FIG. 2, the results of this example reflect a level of controlled release granted through the use of a matrix comprised of a hydrophilic agent and an isoflavone concentrate. This controlled release is shown through a more prolonged period of release of the majority of the contents than the control, as well as a more complete release over the dissolution period. The control B1 released 49% of its contents within the first 3 hours, with a maximum release of 80% of its contents after 24 hours, whereas the matrix formulation B2 released 26% of its contents by hour 3, with a maximum release of 95% of its contents after 24 hours.

TABLE 2

| Dosage | | |
|---|---|---|
| Formulas (mg) | B1 (control) | B2 |
| Isoflavone concentrate | 0 | 125 |
| Isoflavone granules | 377 | 0 |
| HPMC | n/a | 50 |
| Pectin | n/a | 50 |
| NaHCO₃ | n/a | 50 |
| Stearic Acid | n/a | 16 |
| Silica | a/a | 16 |
| Capsule (size 1) | 78 | 0 |
| Total Weight | 455 | 278 |

### Example 3

A monolithic tablet of approximately 818 mg having a hydrophilic agent, a release modifying agent and an isoflavone concentrate was prepared as shown in Table 3. A commercial immediate release tablet of isoflavone concentrate, A1, from Example 1, was used as a control group. In this example, the isoflavone concentrate used is Novasoy™, the hydrophilic agent employed is Hydroxypropyl methylcellulose (HPMC) and the release modifying agent is pectin. Stearic acid is included as a flow agent. Silica is included as a desiccant. Di-calcium phosphate (DCP) is included as a filler and flow agent.

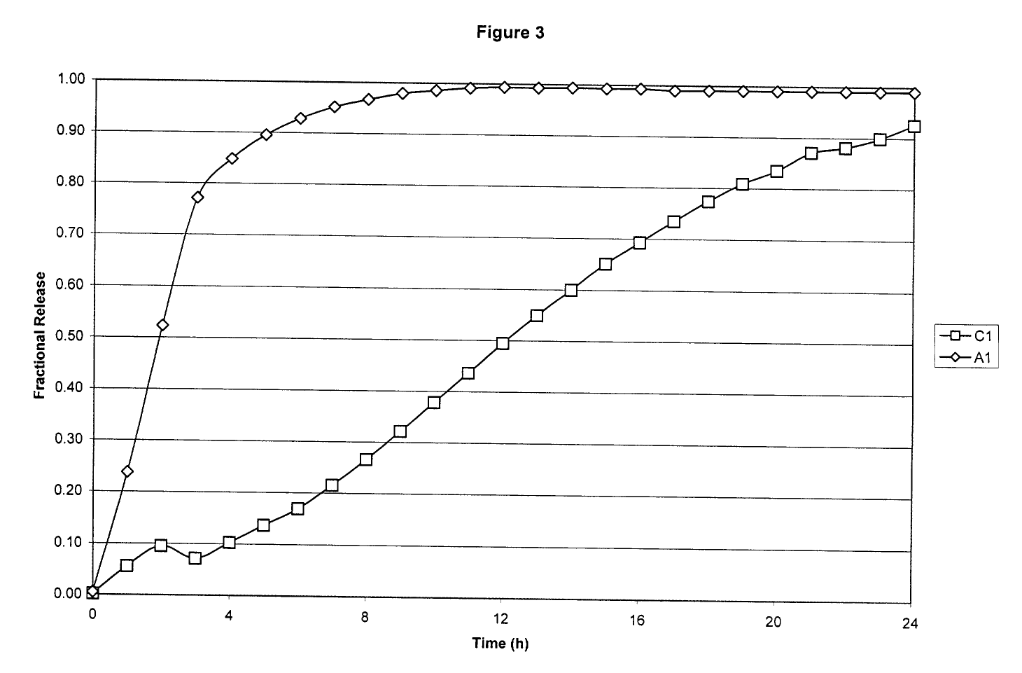

As shown in FIG. 3, the results of this example reflect a level of controlled release granted through the use of a matrix comprised of a hydrophilic agent, release modifying agent and an isoflavone concentrate. This controlled release is shown over an extended duration. Without being bound to a theory, the addition of the hydrophilic agent will retard the release of the isoflavone concentrate from the dosage form. The addition of the release modifying agent further retards the release of the isoflavone concentrate in C1 during the initial 4 hours of matrix hydration, allowing for a more rapid hydration during the intermediate period of 4-19 hours and a more gradual release after 20 hours, yet still allowing 93% complete release after 24 hours.

TABLE 3

| Dosage | |
|---|---|
| Formulas (mg) | C1 |
| Isoflavone concentrate | 250 |
| HPMC | 125 |
| Pectin | 125 |
| DCP | 289 |
| Stearic Acid | 19 |
| Silica | 19 |
| Total Weight | 818 |

### Example 4

A monolithic tablet of approximately 524 mg or approximately 818 mg having a hydrophilic agent, an electrolytic agent and an isoflavone concentrate was prepared as shown in Table 4, with a commercial immediate release tablet of isoflavone concentrate, A1, from Example 1, used as a control group. In this example, the isoflavone concentrate used is NovaSoy™ and the hydrophilic agent employed is Hydroxypropyl methylcellulose (HPMC) and the electrolytic agent is Na₂CO₃. Without being bound to a theory, the addition of the hydrophilic agent will retard the release of the isoflavone concentrate from the dosage form. The addition of the release modifying agent will alter the profile of release and increase the completeness of the release. Stearic acid is included as a flow agent. Silica is included as a desiccant. Di-calcium phosphate (DCP) is included as a filler and flow agent.

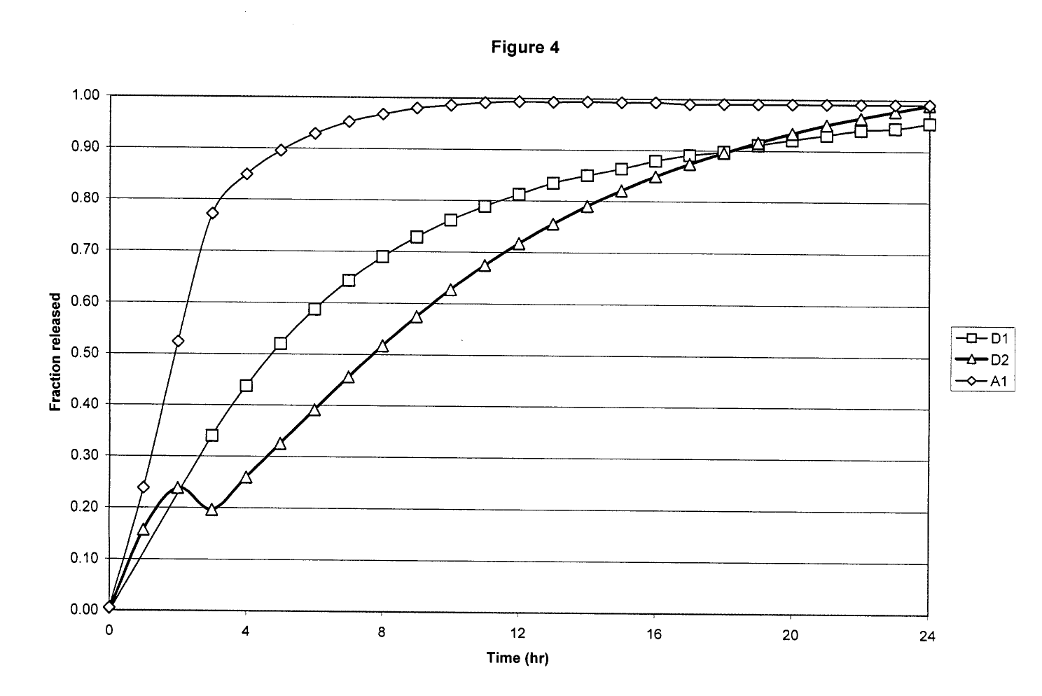

As shown in FIG. 4, the results of this example reflect a level of controlled release granted through the use of a matrix comprised of a hydrophilic agent, electrolytic agent and an isoflavone concentrate. This controlled release is shown over an extended duration. The greater ratio of electrolyte volume to the volume of isoflavone concentrate and total volume of the composition in formulation D2 is reflected in a greater degree of control, with 26% released at 4 hours, 52% at 8 hours, and 72% at 12 hours, in comparison with formulation D1 releasing 44%, 69% and 81% at the 4, 8, and 12 hours, respectively.

TABLE 4

| Dosage | | |
|---|---|---|
| Formulas (mg) | D1 | D2 |
| Isoflavone concentrate | 250 | 125 |
| HPMC | 125 | 75 |
| Na₂CO₃ | 125 | 75 |
| DCP | 280 | 225 |
| Stearic Acid | 19 | 12 |
| Silica | 19 | 12 |
| Total Weight | 818 | 524 |

### Example 5

A monolithic tablet (E1, E2, and E3) of approximately 943 mg having a hydrophilic agent, a release modifying agent, an electrolytic agent and an isoflavone concentrate was prepared as shown in Table 5. A commercial immediate release tablet of isoflavone concentrate, A1, from Example 1, was used as a control group. In this example, the isoflavone concentrate used is Novasoy™ and the hydrophilic agent employed is Hydroxypropyl methylcellulose (HPMC) or Polyethylene Oxide (PEO), the electrolytic agent employed is Na₂CO₃ and the release modifying agent is pectin. The PEO employed is either high (301) or medium (60K) viscosity. The addition of the hydrophilic agent will retard the release of the isoflavone concentrate from the dosage form; the addition of the release modifying agent will alter the profile of release and increase the completeness of the release; and the addition of an electrolytic agent will prevent the hydrolysis of the isoflavones present within the isoflavone concentrate. Stearic acid is included as a flow agent. Silica is included as a desiccant. Di-calcium phosphate (DCP) is included as a filler and flow agent.

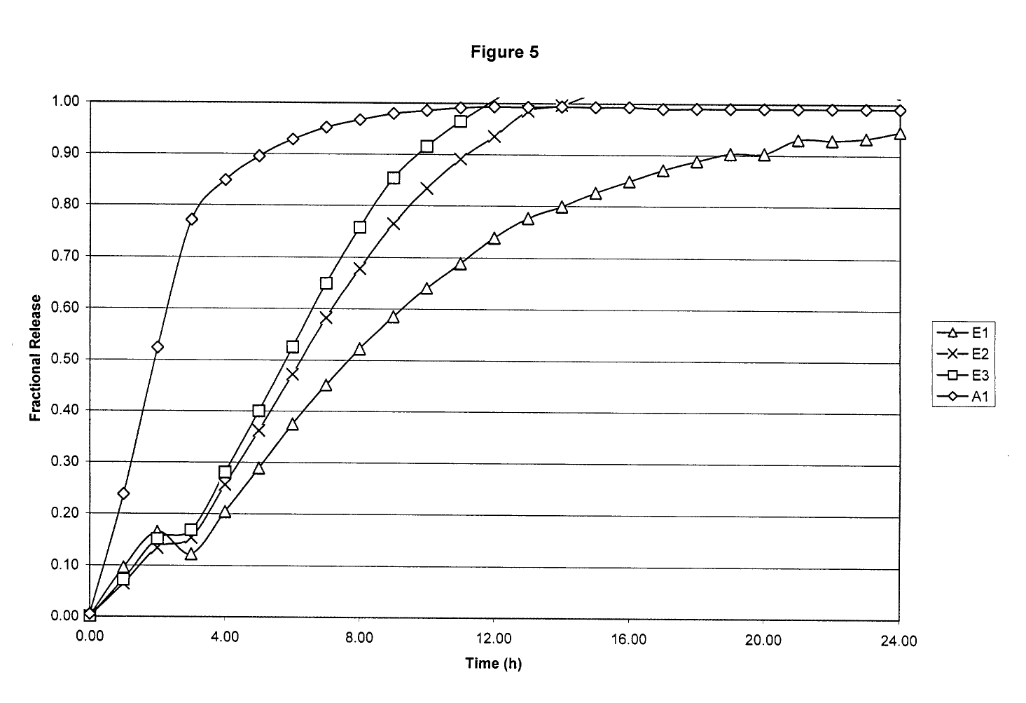

As shown in FIG. 5, the results of this example reflect a level of controlled release granted through the use of a matrix comprised of a hydrophilic agent, release modifying agent, electrolytic agent and an isoflavone concentrate. This controlled release is shown over an extended duration. A superior level of control is demonstrated in HPMC matrices and matrices of high viscosity PEO. Thus, the hydrophilic agent is not limited to a particular type of hydrophilic agent, so long as sufficient matrix viscosity is achieved.

TABLE 5

| Dosage | | | |
|---|---|---|---|
| Formulas (mg) | E1 | E2 | E3 |
| Isoflavone concentrate | 250 | 250 | 250 |
| HPMC | 125 | 0 | 0 |
| PEO 301 | 0 | 125 | 0 |
| PEO 60K | 0 | 0 | 125 |
| Pectin | 125 | 125 | 125 |
| DCP | 280 | 280 | 280 |
| Stearic Acid | 19 | 19 | 19 |
| Silica | 19 | 19 | 19 |
| Total Weight | 943 | 943 | 943 |

### Example 6

A capsule of approximately 344 mg (F2) or approximately 398 mg (F1) was prepared as shown in Table 6, with a commercial immediate release tablet of isoflavone concentrate, A1, of Example 1, used as a control group. The formulations employed both contain a hydrophilic agent, an electrolytic agent, and an isoflavone concentrate, with formulation F2 containing an additional release modifying agent. In this example, the isoflavone concentrate used is Novasoy™ and the hydrophilic agent employed is Hydroxypropyl methylcellulose (HPMC), the release modifying agent is Guar gum and the electrolytic agent is Na₂CO₃. Stearic acid is included as a flow agent. Silica is included as a desiccant. Microcrystalline Cellulose (MCC) is included as a filler and flow agent.

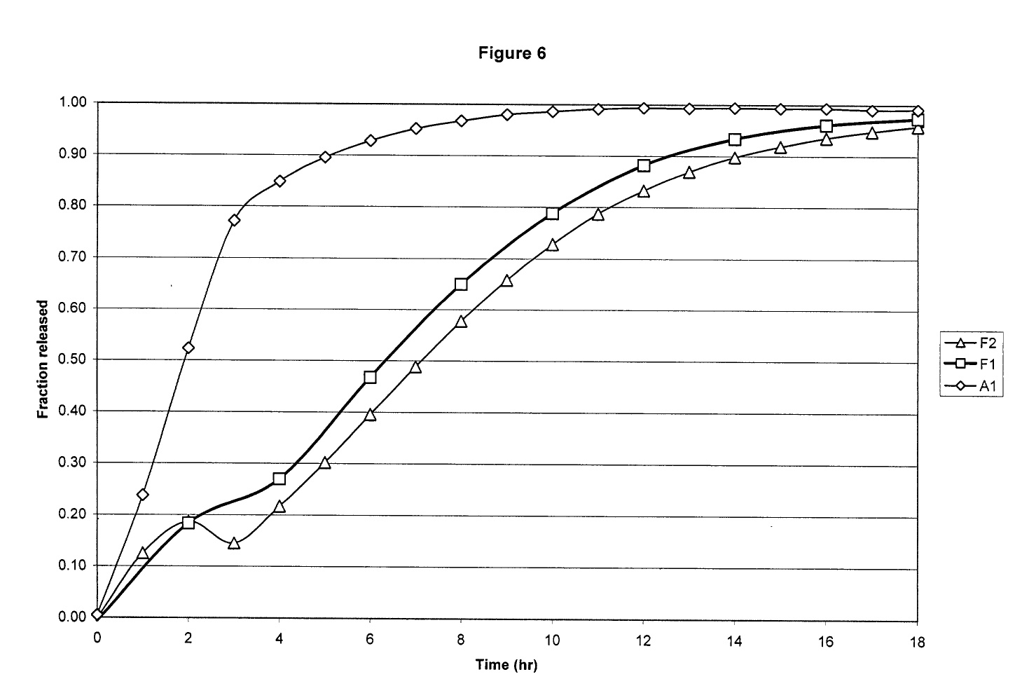

As shown in FIG. 6, the results of this example reflect a level of controlled release achievable in a capsule granted through the use of a matrix comprised of a hydrophilic agent, electrolytic agent and an isoflavone concentrate. This controlled release is shown over an extended duration. Without being bound to any theory, the presence of electrolyte retards the release of the isoflavone concentrate, prevents hydrolysis of the isoflavones present within the concentrate and prevents the premature breach of the capsule.

TABLE 6

| Dosage | | |
|---|---|---|
| Formulas (mg) | F1 | F2 |
| Isoflavone concentrate | 100 | 100 |
| HPMC | 50 | 50 |
| Na₂CO₃ | 50 | 50 |
| MCC | 104 | 0 |
| Guar | 0 | 50 |
| Stearic Acid | 8 | 8 |
| Silica | 8 | 8 |
| Capsule | 78 | 78 |
| Total Weight | 398 | 344 |

### Example 7

A capsule of approximately 760 mg (G1) or approximately 860 mg (G2) was prepared as shown in Table 8, with a commercial immediate release tablet of isoflavone concentrate, A1, as shown in Example 1, used as a control group. The formulations employed both contain a hydrophilic agent, an electrolytic agent, a release modifying agent, an isoflavone concentrate and a Vitamin blend containing Vitamin E, Vitamin C and other vitamins, minerals and nutritional compounds. In this example, the isoflavone concentrate used is Novasoy™ and the hydrophilic agent employed is Hydroxypropyl methylcellulose (HPMC), the release modifying agent is Guar and the electrolytic agent is Na₂CO₃. Stearic acid is included as a flow agent.

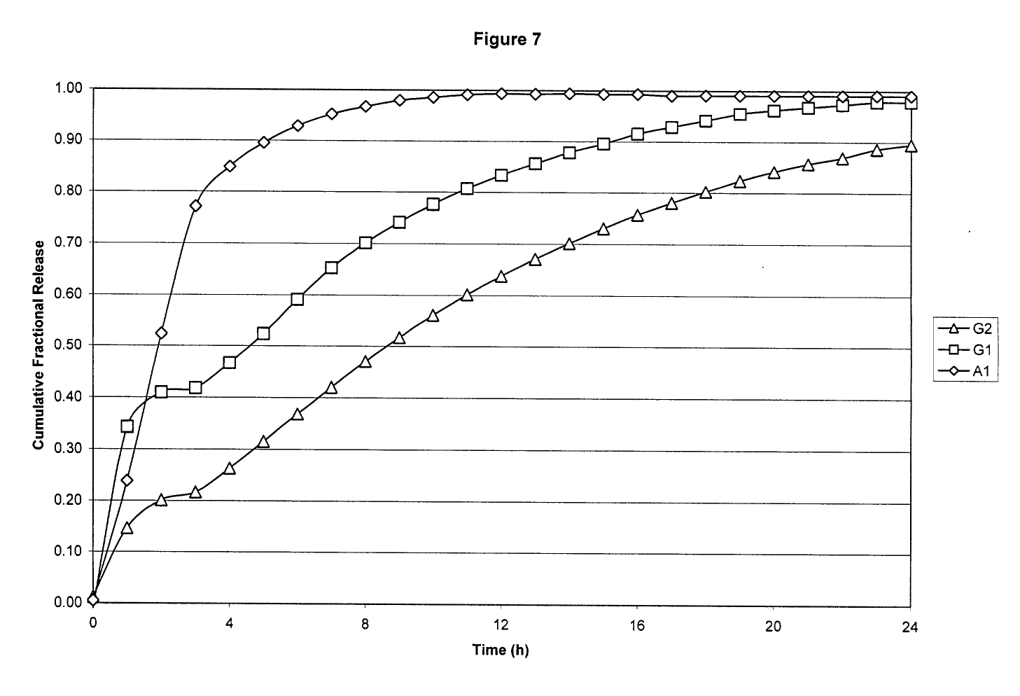

As shown in FIG. 7, the results of this example reflect a level of controlled release achievable in a capsule granted through the use of a matrix comprised of a hydrophilic agent, electrolytic agent and an isoflavone concentrate. This controlled release is shown over an extended duration and in the presence of a substantial volume of vitamin blend. It is likely that the vitamin blend was also delivered in a controlled manner. The capacity to deliver an isoflavone concentrate and a vitamin blend simultaneously is advantageous when employing multiple actives in tandem.

TABLE 7

| Dosage | | |
|---|---|---|
| Formulas (mg) | G1 | G2 |
| Isoflavone concentrate | 100 | 100 |
| HPMC | 50 | 100 |
| Na₂CO₃ | 50 | 100 |
| Guar | 100 | 100 |
| Vitamin blend | 330 | 330 |
| Stearic Acid | 8 | 8 |
| Capsule | 122 | 122 |
| Total Weight | 760 | 860 |

### Example 8

A tablet of approximately 1125 mg (H1) was prepared as shown in Table 8, with a commercial immediate release tablet of isoflavone concentrate, A1, as shown in Example 1, used as a control group. The formulation contains a hydrophilic agent, an electrolytic agent, a release modifying agent, an isoflavone concentrate and a vitamin blend containing Calcium Carbonate, Vitamin E and other vitamins, minerals and nutritional compounds. In this example, the isoflavone concentrate used is Novasoy™, the release modifying agent is pectin, the hydrophilic agent employed is Hydroxypropyl methylcellulose (HPMC) and the electrolytic agent is Na₂CO₃. Magnesium Stearate and Lubritab are included as flow agents. Di-calcium phosphate (DCP) is included as a filler and flow agent.

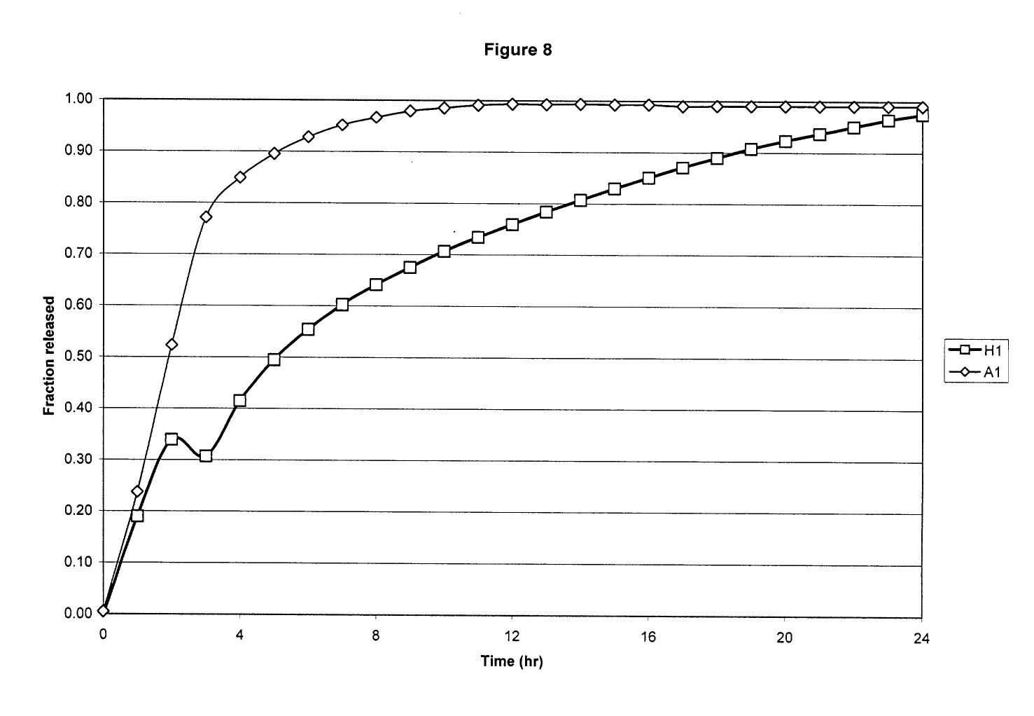

As shown in FIG. 8, the results of this example reflect a level of controlled release achievable in a tablet granted through the use of a matrix comprised of a hydrophilic agent, electrolytic agent and an isoflavone concentrate. This controlled release is shown over an extended duration and in the presence of a substantial volume of vitamin blend. It is likely that the vitamin blend was also delivered in a controlled manner. The capacity to deliver an isoflavone concentrate and a vitamin blend simultaneously is advantageous when employing multiple actives in tandem.

TABLE 8

| Dosage | |
|---|---|
| Formulas (mg) | H1 |
| Isoflavone concentrate | 98 |
| HPMC | 59 |
| Na₂CO₃ | 59 |
| Pectin | 59 |
| DCP | 173 |
| Vitamin blend | 655 |
| Magnesium Stearate | 11 |
| Lubritab | 22 |
| Total Weight | 1125 |

The discussion above is descriptive, illustrative and exemplary and is not to be taken as limiting the scope defined by any appended claims.

We claim:

1. A controlled release delivery system for an isoflavone composition comprising:
a delivery vehicle; and
a delivered component, the delivered component including an isoflavone, wherein delivery of the delivered component is by controlled release.

2. The delivery system of claim 1 wherein the delivery vehicle is a hydrophilic agent selected from at least one of the group consisting of a swellable polymer, polysaccharide, polypeptide, resin, and gum.

3. The delivery system of claim 1 wherein the delivery vehicle is a hydrophilic agent selected from at least one of the group consisting of:
a) a starch selected from the group consisting of rice, corn or potato starch;
b) a gum selected from the group consisting of tragacanth gum, locust beam gum, acacia gum, guar gum, xanthan gum, ghatti gum, or galactomannan gum;
c) an algae derivative selected from alginic acid, sodium alginate, agar, dextran and carageenan;
d) a polysaccharide selected from the group containing pectin and maltodextrin;
e) a cellulose derivative selected from the group consisting of methylcellulose, carboxymethylcellulose, sodium starch glycollate, sodium or calcium carboxymethylcellulose, hydroxyethyl methylcellulose, hydroxypropyl methylcellulose, ethylhydroxy ethylcellulose, ethylmethylcellulose, hydroxyethylcellulose, or microcrystalline cellulose;
f) silica, aluminum silicate, magnesium silicate, aluminum magnesium silicate, sodium silicate or feldspar;
g) a polypeptide selected from the group consisting of gelatin, collagen, casein or heterogeneous protein mixture; and
h) a polymer selected from the group consisting of acrylate, carboxypolymethylene, a polyalkylene glycol or polyvinylpyrrolidone.

4. The delivery system of claim 1 or 2 wherein the delivery system is a monolithic tablet.

5. The delivery system of claim 1 or 2 wherein the delivery system is a capsule.

6. The delivery system of claim 1 or 2 wherein the delivery system is an oral delivery system.

7. A delivery system for an isoflavone composition, the system comprising:
a delivery vehicle;
a release-modifying agent; and
a delivered component, the delivered component including an isoflavone,
wherein the delivery vehicle is a hydrophilic agent.

8. The delivery system of claim 7 wherein the hydrophilic agent is selected from at least one of the group consisting of a swellable polymer, polysaccharide, polypeptide, resin, and gum.

9. The delivery system of claim 7 wherein the hydrophilic agent is selected from at least one of the group consisting of:
a) a starch selected from the group consisting of rice, corn or potato starch;
b) a gum selected from the group consisting of tragacanth gum, locust beam gum, acacia gum, guar gum, xanthan gum, ghatti gum, or galactomannan gum;
a) an algae derivative selected from alginic acid, sodium alginate, agar, dextran and carageenan;
b) a polysaccharide selected from the group containing pectin and maltodextrin;
c) a cellulose derivative selected from the group consisting of methylcellulose, carboxymethylcellulose, sodium starch glycollate, sodium or calcium carboxymethylcellulose, hydroxyethyl methylcellulose, hydroxypropyl methylcellulose, ethylhydroxy ethylcellulose, ethylmethylcellulose, hydroxyethylcellulose, or microcrystalline cellulose;
d) silica, aluminum silicate, magnesium silicate, aluminum magnesium silicate, sodium silicate or feldspar;
e) a polypeptide selected from the group consisting of gelatin, collagen, casein or heterogeneous protein mixture; and
f) a polymer selected from the group consisting of acrylate, carboxypolymethylene, a polyalkylene glycol or polyvinylpyrrolidone.

10. The delivery system of claim 7 wherein the release-modifying agent is selected from at least one of the group consisting of
a) an algae derivative selected from alginic acid, sodium alginate, agar, dextran and carageenan;
b) a polysaccharide selected from the group containing pectin and maltodextrin;
c) a polypeptide selected from the group consisting of gelatin, collagen, casein or heterogeneous protein mixture;
d) a polymer selected from the group consisting of acrylate, carboxypolymethylene, a polyalkylene glycol or polyvinylpyrrolidone; and
e) starch selected from the group consisting of rice, corn or potato starch;
f) a gum selected from the group consisting of tragacanth gum, locust beam gum, acacia gum, guar gum, xanthan gum, ghatti gum, or galactomannan gum;
g) a cellulose derivative selected from the group consisting of methylcellulose, carboxymethylcellulose, sodium starch glycollate, sodium or calcium carboxymethylcellulose, hydroxyethyl methylcellulose, hydroxypropyl methylcellulose, ethylcellulose, ethylhydroxy ethylcellulose, ethylmethylcellulose, hydroxyethylcellulose, cellulose acetate phthalate or microcrystalline cellulose;
h) silica, aluminum silicate, magnesium silicate, aluminum magnesium silicate, sodium silicate or feldspar;
i) aluminum hydroxide; and
j) a polymer selected from the group consisting of acrylate, carboxypolymethylene, a polyalkylene glycol or polyvinylpyrrolidone.

11. The delivery system of claim 7 wherein the delivery vehicle is a monolithic tablet.

12. The delivery system of claim 7 wherein the delivery vehicle is a capsule.

13. The delivery system of claim 7 wherein the delivery system is an oral delivery system.

14. A delivery system for isoflavone composition wherein the system comprises:
a delivery vehicle, the delivery vehicle is a hydrophilic agent;
an electrolytic agent; and
a delivered component, the delivered component including an isoflavone.

15. The delivery system of claim 14 wherein the hydrophilic agent is selected from at least one of the group consisting of a swellable polymer, polysaccharide, polypeptide, resin, and gum.

16. The delivery system of claim 14 wherein the hydrophilic agent is selected from at least one of the group consisting of:
a) a starch selected from the group consisting of rice, corn or potato starch;
b) a gum selected from the group consisting of tragacanth gum, locust beam gum, acacia gum, guar gum, xanthan gum, ghatti gum, or galactomannan gum;
c) an algae derivative selected from alginic acid, sodium alginate, agar, dextran and carageenan;
d) a polysaccharide selected from the group containing pectin and maltodextrin;
e) a cellulose derivative selected from the group consisting of methylcellulose, carboxymethylcellulose, sodium starch glycollate, sodium or calcium carboxymethylcellulose, hydroxyethyl methylcellulose, hydroxypropyl methylcellulose, ethylhydroxy ethylcellulose, ethylmethylcellulose, hydroxyethylcellulose, or microcrystalline cellulose;
f) silica, aluminum silicate, magnesium silicate, aluminum magnesium silicate, sodium silicate or feldspar;
g) aluminum hydroxide;
k) a polypeptide selected from the group consisting of gelatin, collagen, casein or heterogeneous protein mixture; and
l) a polymer selected from the group consisting of acrylate, carboxypolymethylene, a polyalkylene glycol or polyvinylpyrrolidone.

17. The delivery system of claim 14 wherein the electrolytic agent is selected from at least one of the group consisting of a
a) a salt selected from the group containing sodium, calcium, potassium, or magnesium salts;
b) an amino acid; and
c) an ionic compound.

18. The delivery system of claim 14 wherein the delivery vehicle is a monolithic tablet.

19. The delivery system of claim 14 wherein the delivery vehicle is a capsule.

20. The delivery system of claim 14 wherein the delivery system is an oral delivery system.

21. The delivery system of claim 14 wherein the electrolytic agent is capable of inducing an intra-dosage form pH chemically acceptable to preventing the hydrolysis of an isoflavone composition.

22. An delivery system comprising:
a hydrophilic agent;
an electrolytic agent;
a release-modifying agent; and
a delivered component, the delivered component including an isoflavone.

23. The delivery system of claim 22 wherein the hydrophilic agent is selected from at least one of the group consisting of a swellable polymer, polysaccharide, polypeptide, resin, and gum.

24. The delivery system of claim 22 wherein the hydrophilic agent is selected from at least one of the group consisting of:
a) a starch selected from the group consisting of rice, corn or potato starch;
b) a gum selected from the group consisting of tragacanth gum, locust beam gum, acacia gum, guar gum, xanthan gum, ghatti gum, or galactomannan gum;
c) an algae derivative selected from alginic acid, sodium alginate, agar, dextran and carageenan;
d) a polysaccharide selected from the group containing pectin and maltodextrin;
e) a cellulose derivative selected from the group consisting of methylcellulose, carboxymethylcellulose, sodium starch glycollate, sodium or calcium carboxymethylcellulose, hydroxyethyl methylcellulose, hydroxypropyl methylcellulose, ethylhydroxy ethylcellulose, ethylmethylcellulose, hydroxyethylcellulose, or microcrystalline cellulose;
f) silica, aluminum silicate, magnesium silicate, aluminum magnesium silicate, sodium silicate or feldspar;
g) aluminum hydroxide;
h) a polypeptide selected from the group consisting of gelatin, collagen, casein or heterogeneous protein mixture; and
i) a polymer selected from the group consisting of acrylate, carboxypolymethylene, a polyalkylene glycol or polyvinylpyrrolidone.

25. The delivery system of claim 22 wherein the release-modifying agent is selected from at least one of the group consisting of
a) an algae derivative selected from alginic acid, sodium alginate, agar, dextran and carageenan;
b) a polysaccharide selected from the group containing pectin and maltodextrin;
c) a polypeptide selected from the group consisting of gelatin, collagen, casein or heterogeneous protein mixture;
d) a polymer selected from the group consisting of acrylate, carboxypolymethylene, a polyalkylene glycol or polyvinylpyrrolidone; and
e) a starch selected from the group consisting of rice, corn or potato starch;
f) a gum selected from the group consisting of tragacanth gum, locust beam gum, acacia gum, guar gum, xanthan gum, ghatti gum, or galactomannan gum;
g) a cellulose derivative selected from the group consisting of methylcellulose, carboxymethylcellulose, sodium starch glycollate, sodium or calcium carboxymethylcellulose, hydroxyethyl methylcellulose, hydroxypropyl methylcellulose, ethylcellulose, ethylhydroxy ethylcellulose, ethylmethylcellulose, hydroxyethylcellulose, cellulose acetate phthalate or microcrystalline cellulose;
h) silica, aluminum silicate, magnesium silicate, aluminum magnesium silicate, sodium silicate or feldspar;
i) aluminum hydroxide; and
j) a polymer selected from the group consisting of acrylate, carboxypolymethylene, a polyalkylene glycol or polyvinylpyrrolidone.

26. The delivery system of claim 22 wherein the electrolytic agent is selected from at least one of the group consisting of a
a) a salt selected from the group containing sodium, calcium, potassium, or magnesium salts;
b) an amino acid
c) an ionic compound

27. The delivery system of claim 22 wherein the delivery vehicle is a monolithic tablet.

28. The delivery system of claim 22 wherein the delivery vehicle is a capsule.

29. The delivery system of claim 22 wherein the delivery system is an oral delivery system.

30. The delivery system of claim 22 wherein the electrolytic agent is capable of inducing an intra-dosage form pH chemically acceptable to preventing the hydrolysis of an isoflavone composition.

31. An extended release delivery system comprising: about 5% to 40% of hydrophilic agent by total weight; about 5% to 40% of a release-modifying agent by total weight; about 1 to 40% of an electrolytic agent by total weight; and an isoflavone composition.

32. The extended release delivery system of claim 31 wherein the hydrophilic agent is at least one of a cellulose derivative and galactomannan gum.

33. The extended release delivery system of claim 31 wherein the cellulose derivative is hydroxypropyl methylcellulose.

34. The extended release delivery system of claim 31 wherein the release-modifying agent at least one of the group consisting of polysaccharide and a polypeptide.

35. The extended release delivery system of claim 31 wherein the polysaccharide is pectin.

36. The extended release delivery system of claim 31 wherein the electrolytic agent is selected from at least one of the group consisting of sodium carbonate, sodium bicarbonate, sodium phosphate, and calcium carbonate.

37. The extended release delivery system of claim 31 wherein the isoflavone composition is Novasoy®.

38. A pre-dosage form blend of powders, the blend comprising:
about 5% to 40% of an hydrophilic agent by total weight;
about 5% to 40% of a release modifying agent by total weight;
about 1 to 40% of an electrolytic agent by total weight;
and an isoflavone composition.

39. The pre-dosage blend of claim 38 wherein the pre-dosage blend can be formed into a drug delivery system.

40. The pre-dosage blend of claim 39 wherein the drug delivery system is monolithic directly compressed tablet.

41. The pre-dosage blend of claim 39 wherein the drug delivery system is a capsule.

42. The pre-dosage blend of claim 38 wherein the hydrophilic agent is at least one of a cellulose derivative and galactomannan gum.

43. The pre-dosage blend of claim 38 wherein the cellulose derivative is hydroxypropyl methylcellulose.

44. The pre-dosage blend of claim 38 wherein the release-modifying agent at least one of the group consisting of polysaccharide and a polypeptide.

45. The pre-dosage blend of claim 38 wherein the polysaccharide is pectin.

46. The pre-dosage blend of claim 38 wherein the electrolytic agent is selected from at least one of the group consisting of sodium carbonate, sodium bicarbonate, sodium phosphate, and calcium carbonate.

47. The pre-dosage blend of claim 38 wherein the isoflavone composition is Novasoy®.

48. A pre-dosage form blend of powders for a controlled release delivery system, the blend comprising:
an hydrophilic agent;
a release modifying agent;
an electrolytic agent;
a nutritional additive; and
an isoflavone composition.

49. The pre-blend of claim 48 wherein the nutritional additive is selected from the group consisting of at least one of vitamin E, vitamin C, and calcium carbonate.
